(19) United States
(12) Patent Application Publication
(10) Pub. No.: US 2007/0249961 A1
(43) Pub. Date: Oct. 25, 2007

(54) BIOLOGICAL SAMPLE COLLECTION DEVICE

(76) Inventors: Allan D. Morrison, Moorooka (AU); Bradley F. Davin, Seneca, SC (US)

Correspondence Address: KLARQUIST SPARKMAN, LLP, 121 SW SALMON STREET, SUITE 1600, PORTLAND, OR, 97204, US

(21) Appl. No.: 11/676,253

(22) Filed: Feb. 16, 2007

Related U.S. Application Data
(60) Provisional application No. 60/774,372, filed on Feb. 17, 2006.

(51) Int. Cl. A61B 10/02

(52) U.S. Cl. 600/572

(57) ABSTRACT

A swab device for collecting a biological sample from an individual is disclosed, the swab device comprising a head having a swab material for trapping a biological sample thereon and a body comprising means for moving the head relative to the body. Also disclosed is method of obtaining a biological sample from an individual which method comprises contacting at least a portion of the head of a swab device with a surface of the individual, the head of the swab device being moved relative to the body of the swab device whilst the head of the device is in contact with the surface.

### CROSS REFERENCE TO RELATED APPLICATION

This application claims the benefit of priority to U.S. provisional application No. 60/774,372 filed Mar. 17, 2006, the contents of which are herein incorporated by reference.

### FIELD OF THE INVENTION

The present invention relates to swab devices for the collection of biological samples, and the subsequent processing of the swabs.

### BACKGROUND TO THE INVENTION

Biological samples, such as buccal samples are used as the basis for DNA profiling and other diagnostic tests, in many fields, and are typically stored in a dried form on filter paper. Prior to testing, e.g. DNA profiling, a small piece of the sample is punched from the filter paper using either manual punches or punches involving some degree of automation. Current methods of buccal collection are inadequate for a number of reasons.

Typically, samples are collected onto foam-type swabs or other cotton swab material. Samples may be transferred manually to a plain or treated filter paper medium, to allow for the sample's long-term storage and processing. Often, a significant number of the processed samples are found to have insufficient sample material, but only after some processing costs have been incurred.

Further, the process of transferring the sample from the collection device to a media suitable for processing is often time-consuming, and expensive, and not automatable in a cost-effective way, because the collection device has not been designed with a view to automation.

In the case of buccal sampling, where the sample is to be collected directly onto filter paper, it can only be collected onto plain (untreated) filter paper, given that treated filter paper has not been approved by authorities for use in the mouth. In those cases where the buccal sample is collected directly onto plain filter paper, the method of collection does not always ensure that sufficient sample is collected onto the filter paper to allow adequate processing of the sample.

There is also some danger to individuals involved in the collection of buccal samples resulting from the fact that they are required to place their hand adjacent to the mouth of the sample donor, i.e. they are exposed to any infection present in that donated buccal sample, or that could be contracted from the donor.

### SUMMARY OF THE INVENTION

In a first aspect, the present invention provides a swab device for collecting a biological sample from an individual, the swab device comprising a head having a swab material for trapping a biological sample thereon and a body comprising means for moving the head relative to the head, such as means for vibrating and/or rotating the head.

The present invention also provides a swab device for collecting a biological sample from an individual, the swab device comprising a head having a swab material for trapping a biological sample thereon and a mechanism for moving the head relative to the body, such as a mechanism is adapted to vibrate and/or rotate the head.

The movement of the head (e.g. vibration and/or rotation) is intended to improve the transfer of biological material from the individual to the swab material.

In a particular embodiment, the present invention provides a swab device for collecting a buccal tissue sample from the inside of the mouth of an individual, the swab device comprising a head having a swab material for trapping buccal tissue thereon and a body comprising moving the head relative to the head, such as means for vibrating and/or rotating the head.

In one embodiment, the biological sample comprises genetic material, such as DNA.

The swab material may comprise a solid matrix selected from the group consisting of cellulose based materials, hydrophilic polymers, synthetic hydrophilic polymers, polyester, polyamide, plastics (such as polyethylene, polypropylene, polystyrene, polyethylene terephthalate, polytetrafluoroethylene), carbohydrate polymers, fibre glass, porous ceramics and spongy materials, including soft spongy materials.

In one embodiment, at least the portion of the head comprising the swab material is removable from the body.

In a further embodiment, the device comprises a guard interposed between the head portion and a handle portion of the body for avoiding contamination of the handle portion of the body. The guard may be removable or integrally formed with the head and/or at least a portion of the body.

In a related aspect, the present invention provides the use of a swab device of the first aspect of the invention for collecting a biological sample from an individual.

The present invention also provides a method of obtaining a biological sample from an individual which method comprises contacting at least a portion of the head of a swab device of the first aspect of the invention with a surface of the individual, the head of the swab device being moved relative to the body of the swab device, e.g. vibrated and/or rotated, by the body of said swab device whilst the head of said device is in contact with said surface.

The method of the invention may further comprise transferring at least a portion of the sample present on the head of the device to a storage medium by contacting the storage medium with the swab material. In one embodiment, the head of the device is moved relative to the body of the device, such as vibrated and/or rotated by the body of the device when the swab material is in contact with the storage medium.

The storage medium may comprise a composition for protecting genetic material in the sample from degradation.

In a second aspect, the present invention provides a sample cartridge for a swab device which sample cartridge comprises a convex surface formed by a sheet of swab material capable of trapping biological tissue when said convex surface is contacted with a surface of an individual, and a support onto which the swab material is mounted.

The use of a sheet of swab material allows ease of subsequent processing, e.g. punching out of portions of sample by automated means. The convex shape formed by the sheet provides strength to enable the head to withstand the rigours of sample collection from individuals whilst enabling adequate transfer of biological material to the swab material.

In a particular embodiment, the present invention provides a sample cartridge for a swab device which head comprises a convex surface formed by a sheet of swab material capable of trapping buccal tissue when said convex surface is contacted with the inside of the mouth of an individual, and a support onto which the swab material is mounted.

In one embodiment, the support includes a convex rigid supporting member onto or into which the swab material is mounted. The swab support and the swab material may be in the form of a cassette that can be released from the remainder of the cartridge. The supporting member may include one or more apertures.

The cartridge may be reversibly (releasibly) attached to, or mounted on, the remainder of the swab device, in which case the supporting member typically comprises means for reversibly (releasibly) engaging the supporting member with an end portion of a swab device. The terms “releasibly/reversibly attached to/engageable with” mean that the cartridge can be subsequently detached/disengaged from the swab device.

In another embodiment the swab material is in the form of a flexible sheet, and the supporting member is also flexible. In a particular embodiment, the support is capable of adopting a substantially planar configuration and a convex configuration. Again, the supporting member may include one or more apertures.

In another embodiment the swab material is in the form of a flexible sheet, wherein two opposing sides of the sheet are each attached to an elongate member, the elongate members being capable of engaging with the support so as to secure the sheet to the support with the sheet adopting a convex configuration. The elongate members may be hollow.

The swab material may comprise a solid matrix selected from the group consisting of cellulose based materials, hydrophilic polymers, synthetic hydrophilic polymers, polyester, polyamide, plastics (such as polyethylene, polypropylene, polystyrene, polyethylene terephthalate, polytetrafluoroethylene), carbohydrate polymers, fibre glass, and porous ceramics.

In a related aspect, the present invention provides a sample cassette for a sample cartridge of the second aspect of the invention which cassette includes a substantially planar sheet of a swab material capable of trapping biological tissue when said swab material is contacted with a surface of an individual, wherein two opposing sides of the sheet are each attached to a elongate member, the elongate members being capable of engaging the support of said head so as to secure the sheet to the support with the sheet adopting a convex configuration. The elongate members may be hollow.

The present invention further provides a swab device which comprises a sample cartridge as described above, as well as a swab device which comprises a head having a convex surface formed by a sheet of swab material capable of trapping biological tissue when said convex surface is contacted with a surface of an individual, and a support onto which the swab material is mounted.

The present invention also provides a swab device which comprises a head having a convex surface formed by a sheet of swab material capable of trapping a biological sample, such as biological tissue, when said convex surface is contacting with a surface of an individual, and a support onto or into which the swab material is mounted.

The present invention also provides the use of a swab device according to the second aspect of the invention, such as a swab device having a head comprising a sample cartridge or sample cassette according to this second aspect of the invention for collecting a biological tissue sample from an individual.

In a related aspect, the present invention provides a method of obtaining a biological tissue sample from an individual which method includes contacting at least a portion of the head of a swab device with a surface of the individual so as to remove biological tissue, wherein the head of the device is as defined above according to this second aspect of the invention.

The method may further comprise transferring at least a portion of the sample present on the swab material of the device to a storage medium. The storage medium may comprise a composition for protecting genetic material in the sample from degradation.

In another related aspect, the present invention provides a method of transferring a biological sample from a swab to a container for analysis of the genetic material present within the biological sample, which method includes providing a sample cartridge or sample cassette according to the second aspect of the invention, the sample cartridge or sample cassette having a biological sample present on or in the swab material of said cartridge or cassette; arranging said cartridge or cassette in a substantially planar configuration on a substantially planar die plate; and punching out a portion of the swab material so as to transfer said portion to said container.

In a third aspect, the present invention provides a head for a swab device which head includes a plurality of discrete, removable portions of swab material which together form a castellated surface. The uppermost surface of each portion of swab material may have a surface area of from 1 to 30 mm².

Preferably the swab material is a cellulose based material.

In one embodiment each portion of swab material is mounted in the head over a pin which allows each portion to be independently removed from the head by displacement of the corresponding pin.

In a fourth aspect, the present invention provides a head for a swab device which head comprises a layer of porous material capable of trapping cells in contact with a layer of cellulose based material, the layer of porous material being uppermost in use. Preferably the layer of porous material has an average pore size of at least 50 μm. Preferably the layer of porous material has a thickness from 0.5 to 5 mm.

In one embodiment, the head comprises one or more apertures in the layer of spongy material. The one or more apertures may have a diameter of from 2 to 8 mm.

The cellulose based material may comprise a composition for protecting genetic material present in a biological sample collected on said head from degradation.

In a fifth aspect, the present invention provides a head for a swab device, which head comprises a solid support comprising one or more apertures and a layer of swab material overlaying the one or more apertures.

In a related aspect, the present invention provides a head assembly for a swab device comprising (i) a head comprising a solid support comprising one or more apertures and a layer of swab material overlaying the one or more apertures; and (ii) a neck portion.

Preferably the swab device is the swab device of the first aspect of the invention.

In a sixth aspect, the present invention also provides a head for a swab device comprising a swab material, typically provided as a layer, a solid support for the swab material and a cover for the swab material which cover comprises one or more apertures overlaying the swab material and optionally, one or more surface features that assist in removing biological material from a surface and depositing the removed biological material onto the swab material via the one or more apertures.

Preferably the swab device is the swab device of the first aspect of the invention.

### DESCRIPTION OF THE FIGURES

FIG. 1 shows a flexible support which forms part of a cartridge of the invention. The swab material has been omitted for clarity.

FIG. 2 shows the support of FIG. 1 loaded onto the head of a swab device.

FIGS. 3A to 3H show a number of sample cassettes of the invention in cross section and perspective views, the cassettes being in a planar configuration.

FIGS. 4A to 4H show the sample cassettes of FIG. 3 in a curved configuration.

FIG. 5 shows a rigid sample cartridge of the invention with an integral die plate engaged in a punch mechanism.

FIG. 6A shows one embodiment of a rigid sample cassette of the invention.

FIG. 6B shows another embodiment of a rigid sample cassette of the invention.

FIG. 7 shows a sample cartridge and neck portion engaged in a magazine/storage unit. Other sample cartridges stored in the unit are also shown.

FIG. 8 shows a swab device of the invention, complete with powered handle and guard.

FIGS. 9A to 9D show a cross section and perspective view of a swab material of the invention.

FIG. 10 shows an embodiment of a swab device head and neck portion of the invention.

FIG. 11 shows another specific swab device head and neck portion of the invention.

FIG. 12 shows a swab device and storage medium for use in the transfer system described herein.

FIG. 13 illustrates the transfer system described herein and shows a transfer station, swab device and storage unit/magazine.

FIG. 14 shows an embodiment of a swab device head assembly of the invention.

FIG. 15 shows a further embodiment of a swab device head assembly of the invention.

FIG. 16 shows a further embodiment of a swab device head assembly of the invention.

### DETAILED DESCRIPTION OF THE INVENTION

Unless defined otherwise, all technical and scientific terms used herein have the same meaning as commonly understood by one of ordinary skill in the art

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, reference to numerical values, unless stated otherwise, is to be taken as meaning “about” that numerical value. The term “about” is used to indicate that a value includes the inherent variation of error for the device and the method being employed to determine the value, or the variation that exists among the study subjects.

The devices and methods of the invention can be used to collect biological samples, typically from organisms such as animals or plants. Biological samples include physiological/pathological body fluids (e.g. secretions, excretions, exudates) or cell suspensions (e.g., blood, lymph, synovial fluid, semen, saliva containing buccal cells, cervical samples) of humans and animals; and physiological/pathological liquids or cell suspensions of plants. The term “biological tissue sample” refers to a sample that contains cellular material, typically some intact cells.

Preferably the biological samples are samples obtained by contacting the swab device of the invention with a surface of the organism, such as in internal surface e.g. the nasal cavity, the oral cavity, particularly the buccal cavity. Preferably the surface is moist.

In a preferred embodiment the biological sample is obtained non-invasively.

In some embodiments, e.g. the collection samples from relatively dry surfaces such the skin, it may be desirable to wet the swab materials described below prior to sample collection.

In one embodiment the biological sample contains genetic material. As used herein, the phrase “genetic material” means either or both deoxyribonucleic acid (DNA) or ribonucleic acid (RNA). However, the present invention is not restricted to sampling and testing of nucleic acids.

Swab Materials

The head of the swab devices of the invention include a swab material that is capable of trapping a biological material thereon. For example the biological material may be adsorbed or absorbed by the swab material and/or entrapped within the material.

Typically the swab material includes a dry solid medium. In one embodiment, the swab material has a contiguous surface e.g. it is not in the form of bristles or hairs. The contiguous surface preferably has a surface area available for contact with a biological sample of at least 1 mm², such as at least 2, 3, 4 or 5 mm².

In a particular embodiment, the swab material is substantially planar or is capable of adopting a substantially planar configuration e.g. it is in the form of a sheet which may be flat or curved.

In one embodiment the dry solid medium provides for both capture of the biological sample and storage and/or subsequent analysis of the stored sample. This is referred to herein as the direct application form or embodiment of the invention. In another embodiment, the dry solid medium simply serves to capture the biological sample during the sampling process with the sample being transferred subsequently to a storage medium. This is referred to herein as the transfer form or embodiment of the invention.

Suitable materials for the dry solid medium include, but are not limited to, a matrix which is cellulose based (e.g., cellulose, nitrocellulose or carboxymethylcellulose papers), hydrophilic polymers including synthetic hydrophilic polymers (e.g., polyester, polyamide, carbohydrate polymers), plastics (such as polyethylene, polypropylene, polystyrene, polyethylene terephthalate, polytetrafluoroethylene, e.g. Empore.™., 3M, St. Paul, Minn.), fibreglass and porous ceramics. Cellulose based materials may, for example, be derived from plant materials, e.g. cotton and derivatives thereof. Other materials include spongy materials e.g. foams, such as polymer foams including porous (poly) urethane foams, and polyurethane open cell foams.

Typically, the layer or layers of porous material are relatively thin, preferably having a total thickness of less than 4 or 5 mm, such as from 0.1 to 5 mm, preferably from 1 to 3 mm, typically at least 0.3 or 0.5 mm.

In one embodiment, the swab material is selected so that it is suitable for storage of the collected biological sample. For example, the dry solid medium may further comprise a composition that can protect against degradation of genetic material stored on the solid medium. The composition may optionally also cause inactivation of microorganisms which may be associated with a biological sample which may be potentially pathogenic to human handlers of the stored sample of genetic material.

The composition may include one or more of a weak base, a chelating agent, or any anionic detergent or surfactant. In addition, the composition may also include uric acid or a urate salt. Suitable compositions are described in more detail in U.S. Pat. No. 6,627,226.

In another embodiment of the invention, the dry solid medium may further include a component which is functional in the subsequent analysis to be performed on the stored genetic material. Subsequent analysis which may be performed on a sample of genetic material stored on the dry solid medium includes analysis methods known in the art, for example, polymerase chain reaction (PCR), ligase chain reaction (LCR), reverse transcriptase initiated PCR, DNA or RNA hybridization techniques including restriction fragment length polymorphism (RFLP) and other techniques using genetic or DNA or RNA probes, genomic sequencing, enzymatic assays, affinity labelling, method of detection using labels or antibodies and other similar methods. In a preferred embodiment, the dry solid medium of the invention is a suitable medium for storage of components for subsequent analysis, which are included on the dry solid medium.

In the case of stored RNA, particularly unstable RNA, components for subsequent analysis that may be included may also provide protection against RNA degradation. This includes RNase inhibitors and inactivators, proteins and organic moieties that stabilize RNA or prevent its degradation.

Whilst the swab material is typically supplied as a dry matrix, it may be desirable in some applications of the material to be supplied pre-wetted. For example, when collecting samples from the skin of an individual, which is essentially dry, wetting of the material can assist in the removal of cells. This could also be achieved by wetting separately the skin, or other surface from which it is desired to take a sample, prior to contacting the swab material.

In one aspect, the present invention is directed to a new construction of sample collection media, comprising one or more layers of a biological sample storage material, e.g. a cellulose based material, such as filter paper, overlaid by one or more layers of a porous material, such as a sponge-type material. In use, at least one layer of the porous material will be uppermost to the sample surface. In one embodiment it may completely enclose the sample storage material. This porous material is preferably suitable for use in the human mouth. Typically, it is also compatible with subsequent assay procedures, such as nucleic acid amplification and hybridisation procedures, in circumstances where it is likely or possible that any portion of it may be removed from the head/cartridge with the cellulose based material, and become involved in the further processing stages.

Examples of some suitable materials include polymer foams such as some types of porous urethane foam, and polyurethane open cell foam.

The porous material is generally selected so that it is capable of capturing cellular material, i.e. whole cells. Preferably the porous material has an average pore size of at least 20 μm, more preferably at least 50 or 100 μm. Typically the average pore size is less than 1 mm, such as less than 500 or 200 μm.

Typically, the layer or layers of porous material are relatively thin, having a total thickness of less than 5 mm, such as from 0.1 to 5 mm, preferably from 1 to 3 mm, typically at least 0.3 or 0.5 mm.

Preferably the sample storage material is a dry solid medium as described above.

Typically, the layer or layers of sample storage material have a total thickness of less than 5 mm, such as from 0.1 to 5 mm, for example from 0.3 or 0.5 mm to 5 mm.

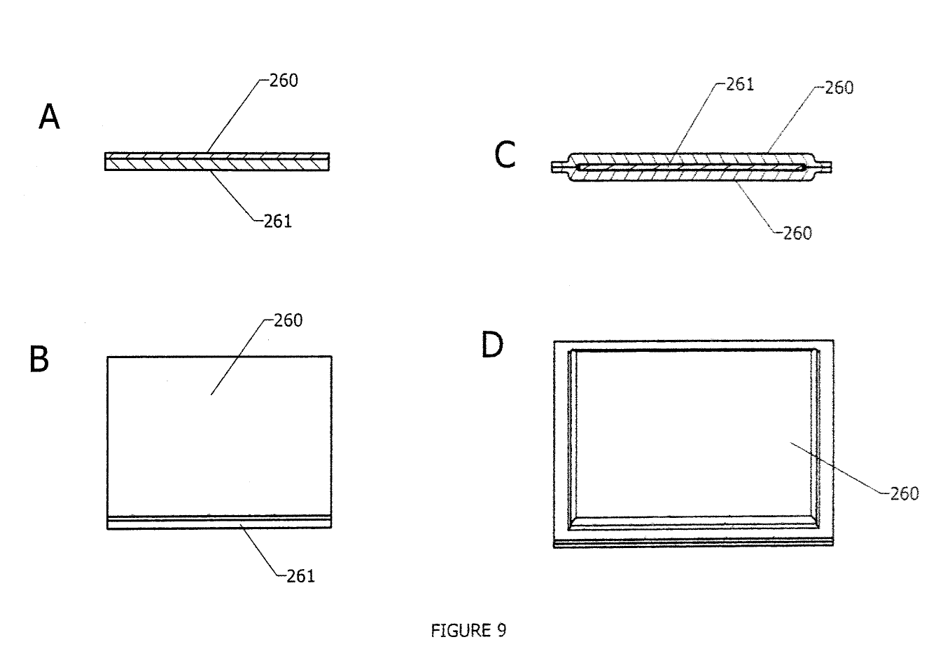

With reference to FIGS. 9A and 9B, in one embodiment, the material has a single layer of porous material (260) on top of a single layer of a sample collection material e.g. filter paper (261).

With reference to FIGS. 9C and 9D, in another embodiment, the material comprises a layer of porous material (260) on each side of a layer of sample collection medium (261), to maximize the efficiency of the device in collecting cells, and at the same time adding strength to the material. The layers may be joined to seal the sample collection medium within.

When used to collect buccal samples or the like, the use of the porous material allows for the collection of cell material, which is then transferred to the cellulose based material below by laminar flow. At the same time the layering of the porous material and filter paper provides additional strength to the construct, such as when the swab material is a curved sheet and is pressed against the inner cheek.

A further advantage in some embodiments is that the porous material can allow for the use of storage media that are not approved for contact with humans or animals since the porous material separates the individual from the storage medium during sampling. Consequently, even though the porous material may be wetted on contact with the individual, collection of sample onto or into the porous material can occur before any fluid in the sample can cause transfer of substances in the storage medium back towards the surface of the porous layer where it could potentially be transferred to the individual from whom the sample is being taken. Thus, in embodiments where it is desired for the storage medium to include compositions that, for example, preserve the integrity of genetic materials, but which are not permitted to be in contact with humans or animals, the swab material of this aspect of the invention allows direct transfer and storage of samples without the need for transfer of the sample to a separate storage medium.

In a particular embodiment, the porous material comprises one or more apertures, such as a plurality of apertures. These are intended to allow for punching out sections of the filter paper below for further processing without the spongy material.

The apertures will generally be compatible with, and/or correspond to, the size of sample that it is desired to punch out. For example, the one or more apertures may have a width or diameter of from 1 to 10 mm, such as from 2 to 8 mm. As a guide, the width of diameter of the apertures will be at least 1 or 2 mm greater than the width or diameter of the punch used to punch out samples.

The sample storage material may include a composition for protecting genetic material present in a biological sample collected on the material from degradation, as described above.

Where the sample collection medium is used in conjunction with a swab device which comprises means/a mechanism for moving, e.g. oscillating, vibrating and/or rotating, the head of the device relative to the body of the device, as described below, it may be desirable after sample collection to position the head horizontally with the porous layer uppermost and then operate the device to begin movement, e.g. oscillation/vibration/rotation, of the head to assist in transfer of biological material through the porous layer and onto the sample storage material.

Swab Devices

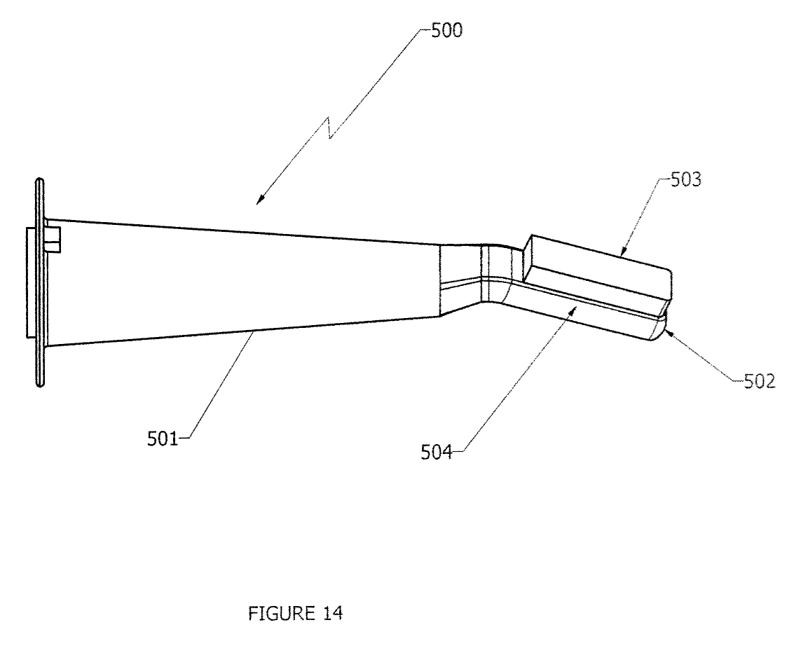

In its broadest form, a swab device of the invention comprises a head which has a swab material capable of trapping a biological sample thereon and a body which enables the user and/or machinery to grip the swab device and thereby manipulate the device when obtaining a sample. The body of the device may optionally be divided into two portions: a handle and a neck portion, the neck portion generally being removable from the handle. The neck portion may be integrally formed with at least a portion of the head or it may be separately formed and detachable from the head. Accordingly, various combinations are possible including: a device where the head and body are integrally formed; a device where the head and body are separate; a device with a head, neck portion and handle where the head and neck portion are integrally formed and the handle is removable; a device with a head, neck portion and handle where all three components are separate. In one embodiment, the head is at an oblique angle to the neck (see FIGS. 14 to 16), such as an angle of 135 to 175 degrees.

Various heads, head assemblies, samples cartridges and sample cassettes that are suitable for use with the swab device are described below.

In addition, a further optional component is a guard, as described in more detail below, for protecting all or part of the body/handle from contamination. In one embodiment the guard is a separate component, to be fitted between the head and the body, or between the neck portion and the handle. In another embodiment, the guard is integrally formed with the head, neck portion or body.

The invention is directed to a number of aspects. In a first aspect, the body of the device is provided with means for moving the head of the device relative to the body of the device, such as means for vibrating and/or rotating the head of the device. The rapid movement, e.g. oscillating, vibration and/or rotation, of the head of the device enables improved collection of biological material. Typically the device can be used to collect cellular material from a surface of an individual, preferably an internal surface such as the buccal cavity or nasal cavity.

The means for moving the head relative to the body e.g. for vibrating and/or rotating and/or oscillating and/or providing a reciprocating motion, may comprise an electric motor within the housing of the body of the device. A convenient means for vibrating and/or rotating the head of the device may be provided in the form of a conventional battery powered electric toothbrush. However it will be appreciated that any suitable arrangement can be used. It will also be appreciated that the movement of the head should be sufficiently rapid to enable effective collection of the biological sample e.g. at least 5, preferably at least 10, movements per second. In some embodiments, the movements may be in the order of hundreds of movements per second, such as at least 100, 200 or 500 movements per second.

Typically, the head of the device, on which the sample is to be collected, is detachable from the body of the device so that the body of the device can be reused.

In one embodiment, the device further comprises a hand guard which is designed to reduce or minimise the transfer of biological material from the individual whom it desired to take a sample from to the operator of the device and/or the handle of the device. Accordingly, the guard is interposed between at least a portion of the body (e.g. the handle portion) and the head. The guard may be of any suitable shape and size necessary to perform the function of protecting the hand of the operator and/or at least a portion of the body of the device from contamination. The guard may be detachable from the body and/or the head. Thus in a preferred embodiment the device comprises at least three separate detachable components, namely the handle, the guard and the head.

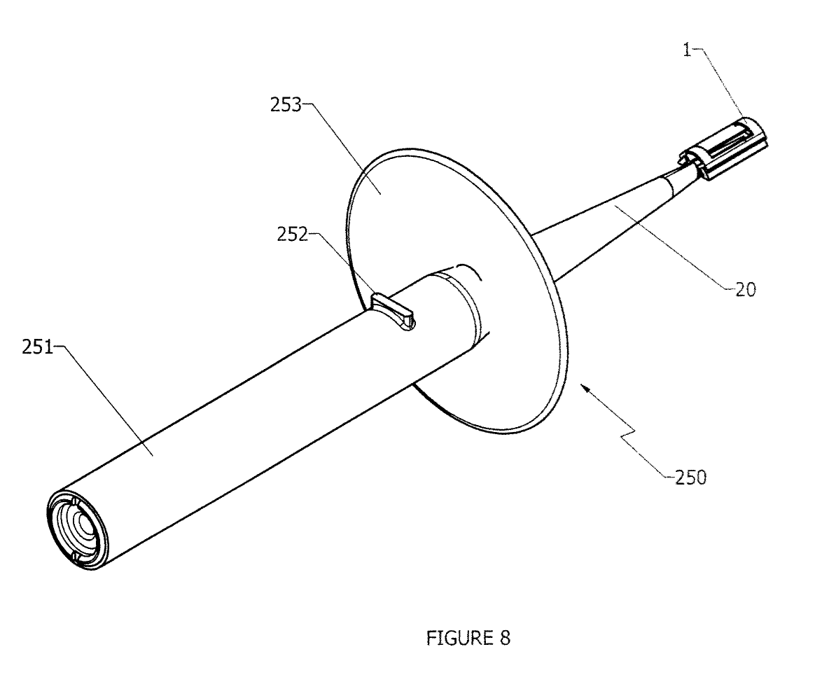

Referring to FIG. 8, the swab device (250) comprises a powered handle (251), such as in the form of an electric toothbrush with an on/off switch (252). Attached to the top of the handle is a hand guard (253). A neck portion (20) engages with the upper end of the handle. The other end of the neck portion is attached to a head (1) which includes a sample cartridge containing a swab material, such as is described in more detail in relation to the second aspect of the invention.

In use, the head (1), containing swab material, is placed against the side of the buccal cavity of an individual and the on/off switch (252) pressed to begin the movement, e.g. oscillation/vibration/rotation, of the head against the inside of the individual's mouth. When sufficient time has elapsed to ensure capture of the biological sample, the on/off switch (252) is again operated to stop the movement, e.g. vibration/rotation, of the head and the device is removed from the individual's mouth. The head containing the sample can then be processed as described below, i.e. directly or following transfer of the biological material to another medium such as a storage medium.

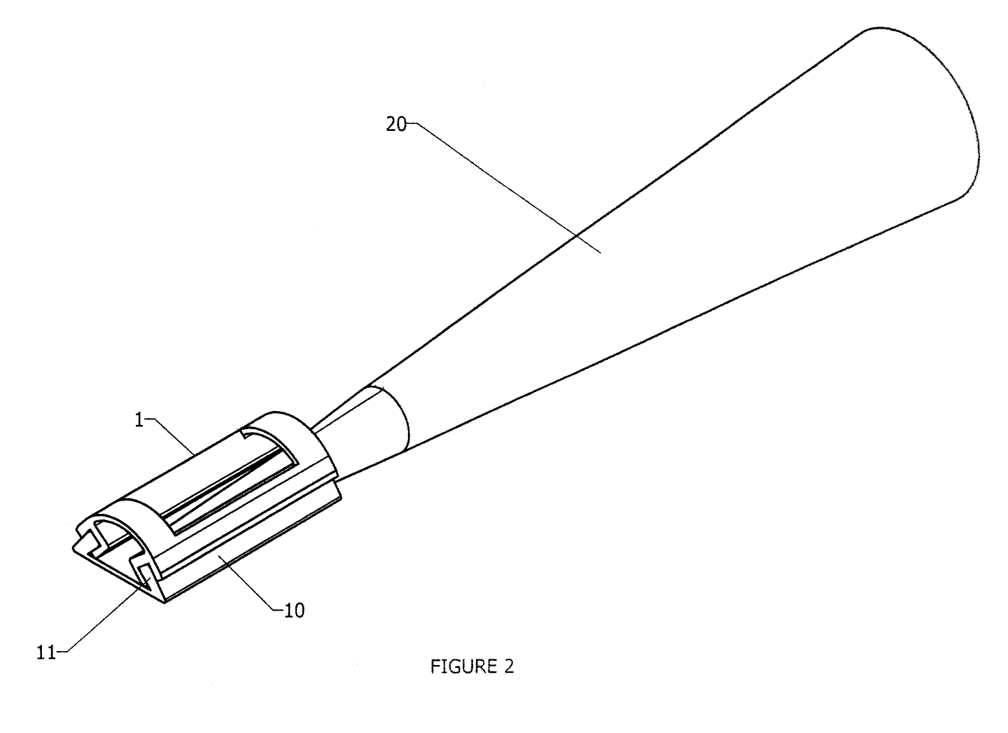

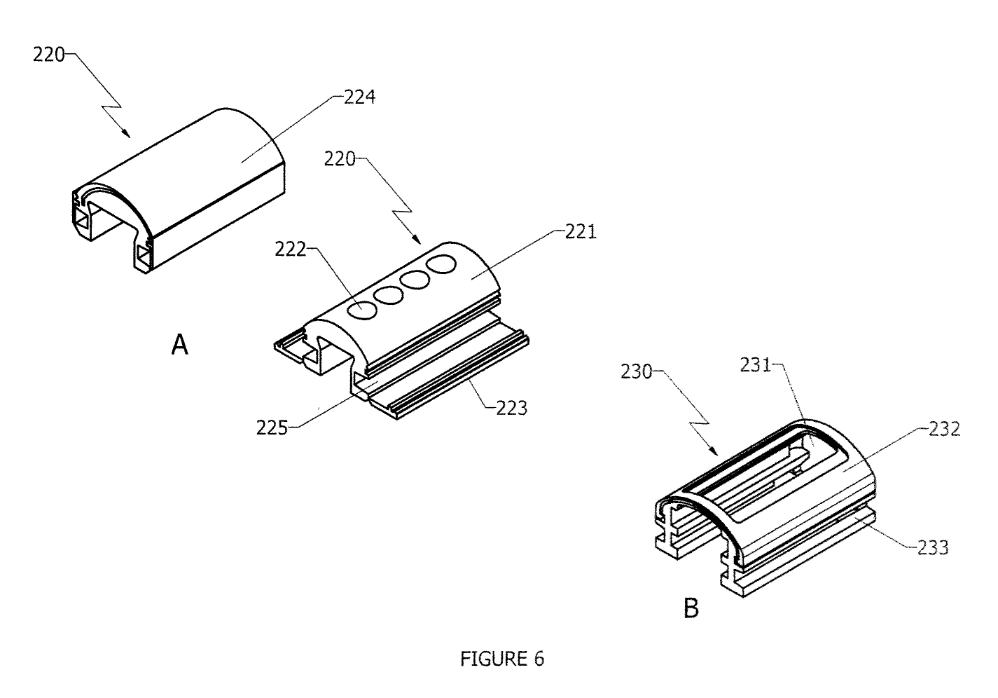

The second aspect of the invention is directed to the head of the swab device. The head, or a portion thereof, may be integrally formed with other components of the swab device, such as the body or a portion therefore, for example a neck portion, or it may be detachable from other components of the swab device. In some embodiments, the head includes fixed swab material for the collection of samples. In other embodiments, such as are described in FIGS. 2 and 6, the swab material is part of a cartridge or cassette which can be separated from the remainder of the head. The heads, head assemblies, sample cartridges and sample cassettes described herein are typically suitable for use with the swab device described above.

The head is generally described hereinafter in relation to embodiments where the head comprises a removable sample cartridge or sample cassette, but it is to be understood that where applicable, features and embodiments equally apply to a fixed head arrangement where the swab material is designed to be fixed to the head.

In certain embodiments, the head, or a portion thereof, may be designed to be fixed to and manipulated with an end portion of the body of the device termed a “neck portion”. The neck portion can generally be detached from the remainder of the body, such as a reusable handle so that the neck portion and head can be subsequently processed together. The neck portion is preferably formed such that when detached from the remainder of the body of the device it can be held either by a user or a machine used to manipulate samples.

In a particular embodiment, the neck portion is integrally formed with at least a portion of the head. This includes variants where the head includes fixed sample media as well as variants where the head is designed to be used with removable cartridge type media. For example, in the cartridge embodiments described below, the support of the cartridge could also be provided as an integral part of the head of the device.

In an alternative embodiment, the neck portion is separately formed and can be detached from the head, especially for subsequent automated punching.

The head is preferably formed such that the swab material is in the form of a sheet that can adopt a convex configuration for the purpose of obtaining a sample. The curved configuration provides the sheet with sufficient strength to remain intact when contacting a sample surface, with pressure applied, such as the inside of an individual's mouth, whilst the use of a sheet of material simplifies subsequent direct processing of the sample without the need to transfer the biological material to another medium.

Referring to FIGS. 6A and 6B, in one embodiment, the sheet of swab material (224) is supported on a rigid support (220, 230)) which has a convex portion (221) which corresponds to the convex configuration of the sheet or enables the sheet to adopt a convex configuration. The rigid support preferably has one or more apertures (222, 231), such as a plurality of apertures, aligned with the swab material to enable subsequent punching out of the portions of swab material containing biological material for processing. Preferably the apertures have a surface area of at least 1 mm², such as from 1 to 30 mm². The apertures preferably have a width or diameter of at least 1 mm, such as from 1 to 6 mm. As a guide, the width or diameter of the apertures may be selected so as to be about 1 to 2 mm greater than the width or diameter of the punch subsequently used to punch out samples.

In one embodiment, the rigid support acts as a die plate for subsequent punching out of samples (FIG. 6B).

The rigid support may include engagement means for securing the sheet of swab material to the support, for example in the form of an elongate clamp at either side of the support (223). In one embodiment, the sheet is secured by a cover (232) which clips over the sheet and engages the sides of the support. The cover may contain one or more apertures (231) to permit contact between the swab material and the biological sample during the sampling process. Alternatively, the swab media can be fixed to the support using adhesive, or by any other suitable means.

The base (223, 233) or an end of the rigid support may be shaped so as to engage reversibly with a portion of the head of the swab device, i.e. adapted to engage with a portion of the head of the swab device. The shape of the base (223, 233) or an end of the rigid support may also serve to engage reversibly the support with the die plate of a punch apparatus and/or with a locating means in a storage magazine for storing a plurality of the heads/cartridges, as will be described in more detail below. In other words, the support may be designed so that the sample cartridge can be securely grasped and held by the swab device and optionally any other devices required to manipulate the sample in other stages of sample processing. For example, the base may include a longitudinal groove (223, 233) which cooperates with a suitably shaped portion of an end portion of the head of the swab device. An alternative is for the support to include hollow portions into which engagement members (e.g. arms) on the head of the swab device or other component of the system can be inserted.

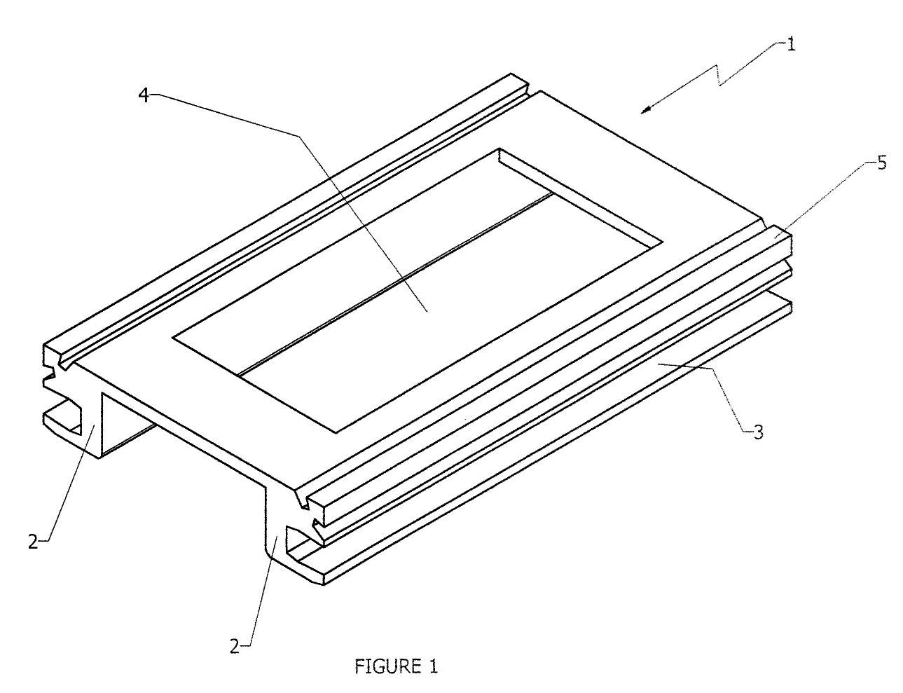

Referring to FIGS. 1 and 2, in another embodiment, the support of the cartridge (1) is flexible such that it can adopt a convex configuration (see FIG. 2) but is also capable of adopting a substantially planar configuration (see FIG. 1). This has the advantage of improved sample capture in the convex configuration as described above whilst allowing the sample to be processing in a flat configuration. The flexible support is also referred to hereinafter as a sample cartridge.

The flexible support may include engagement means (5) for securing the sheet of swab material to the support, as described above in relation to the rigid support. The flexible support preferably includes one or more apertures (4) as described above in relation to the rigid support.

The base of the flexible support may be shaped so as to reversibly engage with an end portion of the swab device, such as the neck portion (20) of the swab device. FIG. 2 shows the sample cartridge of FIG. 1 engaged with a portion (10) of the head, which is in turn attached to a neck portion (20). The head portion (10) includes elongate members (11) which engage with the grooves (3) in the base of the sample cartridge. The spacing of the elongate members (11) is less than that of the grooves (3) which results in the flexible support (1) being forced into a convex configuration. However, when the flexible support (1) is removed, it can return to a planar configuration.

The shape of the base of the flexible support may also serve to engage reversibly the support with the die plate of a punch apparatus and/or with a locating means in a storage magazine for storing a plurality of the heads/cartridges, as described above in relation to the rigid support.

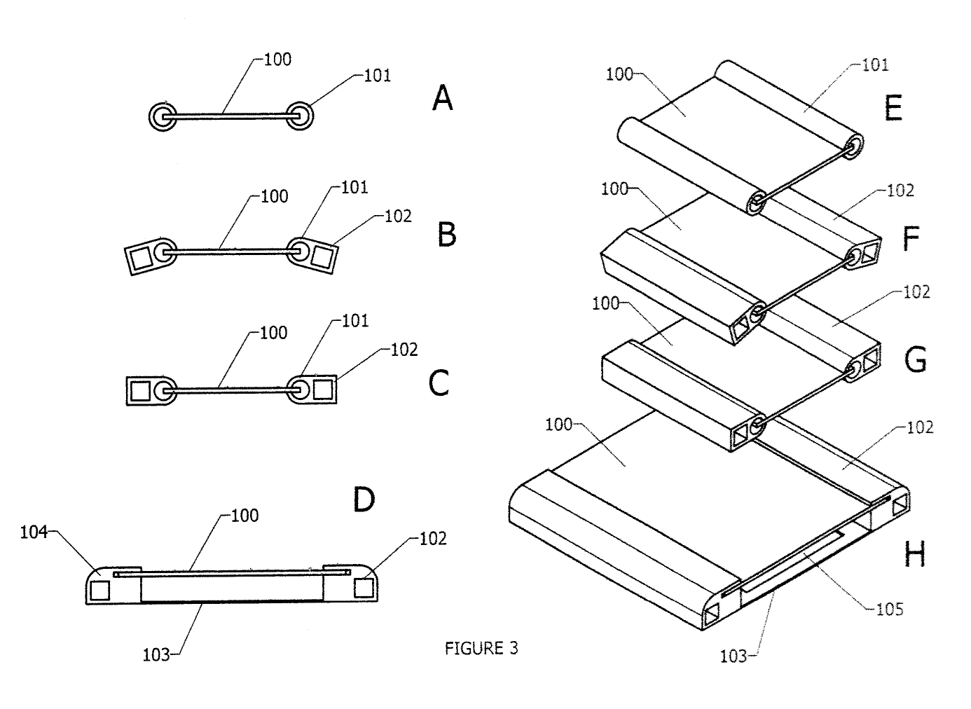

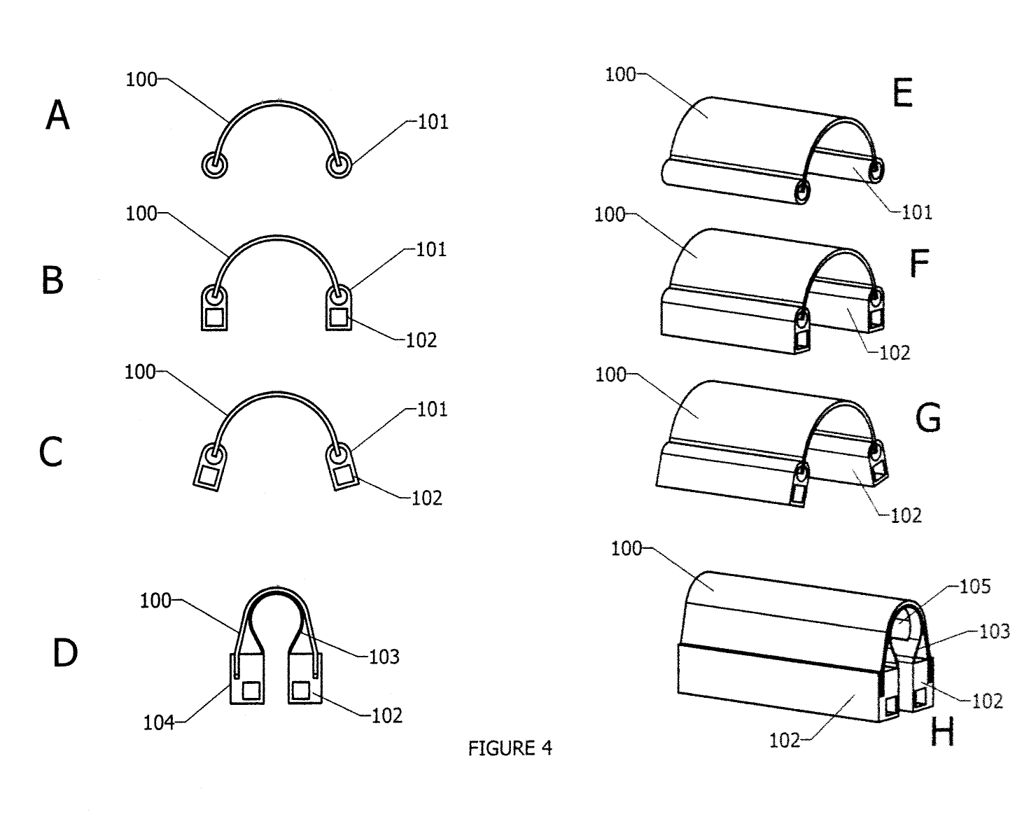

Referring to FIGS. 3 and 4, in another embodiment, a sheet of swab material (100) is attached on each of two opposing sides to an elongate member (101, 102) which serves to engage with the remainder of the swab device in a similar manner to that described above for the flexible support. The elongate member serves to reversibly engage with the remainder of the swab device, e.g. a portion of the head (10) as shown in FIG. 1, and/or other elements of the system such as the sample punch or a sample magazine. Typically, the elongate members are joined to one another via the sheet of swab material and by any other connecting means.

For simplicity, this embodiment and the related embodiments described below are termed a sample cassette.

In one embodiment, the elongate members (101) include a hollow section (102) which can reversibly/releasibly engage with the remainder of the swab device and/or other elements of the system.

In a similar manner to the flexible support embodiment, these sample cassettes can be assembled onto the remainder of the swab device such that the sheet of swab material adopts a convex configuration (see FIGS. 4A to 4H).

In one embodiment shown in FIGS. 3d, 4d the elongate members (104) are joined by a flexible support sheet (103) which lies below the swab material. When the sample cassette is assembled onto the head of the swab device, the flexible support sheet (103) contacts the underside of the swab material (100) to provide additional structural support to the swab material (100) during the sampling process.

In general, to maximise the collection of cellular material, the media described in the various embodiment should maintain enough pressure on the collection surface, e.g. the buccal lining to scrape the cellular material onto its surface. The pressure exerted on the surface is dependent on the rigidity of the media—media support (sample collection) structure. The rigidity of the curved swab material is partly determined by the curvature of the swab material (and where present, the support onto which the swab material is fixed).

In some embodiments, particularly where the curved swab material does not have any supporting structure in contact with the upper portions of the material, i.e. at or near the midpoint of the curve, it is preferred that the height of the curved swab material is preferably less than 20 mm, such as less than 15, 10 or 5 mm. The height of the curved material is measured as the perpendicular distance from the midpoint of the curved swab material to the midpoint of the base of the material—a.

The width of the base of the curved sheet of swab material b in the various embodiments of the second aspect of the invention is preferably from 5 to 20 mm, such as from 5 to 15 mm when the swab material is in the convex configuration ready for the taking of a sample.

In the various embodiments described above, it is preferred that when the swab material adopts a convex configuration in use such that the ratio of the height of the curved sheet of swab material a, to the width of the base of the curved sheet of swab material b is from 1:2 to 7:4, such as from 2:3 to 3:2. Expressed as the angle φ between b and the hypotenuse c of a right angle-triangle having as its third side a, φ is typically from about 10 to 60 degrees, such as from 20 to 50 degrees e.g. 40 to 50 degrees. In one embodiment, the convex curve is a slight curve e.g. φ is typically from about 5 to 25 degrees, such as from 10 to 20 degrees.

In certain embodiments, the swab material is covered in use by a cover which contains one or more, typically a plurality of apertures. The upper surface of the cover which in use contacts a surface of the individual from whom it is desired to obtain a biological sample, may be formed, e.g. with ridges, dimples or blades, so as to assist with scraping biological material, particularly cellular material, from the individual. The apertures are typically formed adjacent to the surface features, e.g. the ridges, dimples or blades, so as to allow the removed biological material to be deposited onto the swab material. The cover may be rigid or flexible and may be fixed to the remainder of the head by any suitable means, such as by snap fitments. The cover may for example be made of a plastic material such as a polymer selected from polyethylene, polypropylene, polystyrene, polyethylene terephthalate and polytetrafluoroethylene.

Accordingly, the present invention also provides a head for a swab device comprising a swab material, typically provided as a layer, a solid support for the swab material and a cover for the swab material which cover comprises one or more apertures overlaying the swab material and optionally, one or more surface features that assist in removing biological material from a surface and depositing the removed biological material onto the swab material via the one or more apertures.

The cover may include a flap or other type of valve mechanism that prevents liquid and cells passing back from the swab material through the apertures to the surface from which the swab is being taken.

The heads/sample cartridges/sample cassettes of the second aspect of the invention can, for example, be supplied individually wrapped and ready to assemble onto the swab device. Alternatively, they can be supplied pre-assembled onto an end portion of the swab device (e.g. a neck portion), particularly where used with a handle as described in the first aspect of the invention, or pre-assembled with the remainder of the swab device. For example where a rigid support is used, the support may be integral with one or more components of the remainder of the swab device, such as a head portion, or even the complete swab device.

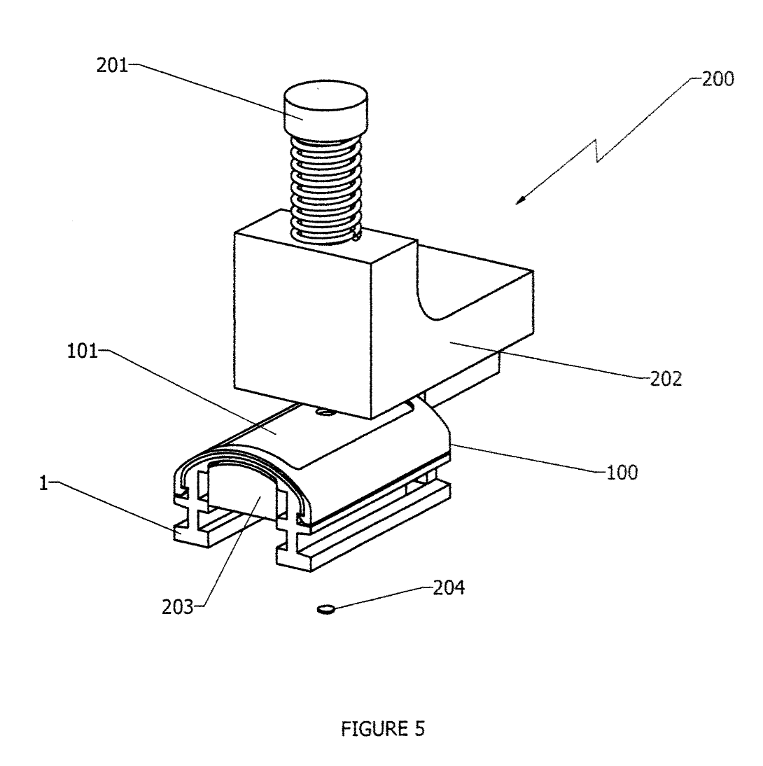

In a preferred embodiment, the heads/sample cartridges/sample cassettes of the second aspect of the invention can be removed from at least the handle portion of the swab device.

Where the swab material is to be used directly for sample processing, i.e. without transfer to a separate medium, once the swab device has been used to obtain a biological sample, the head/sample cartridge/sample cassette can be transferred to a device suitable for removing one or more portions of the swab material for further processing/storage, such as a punch. FIG. 5 shows a sample cartridge (100) which has been detached from the remainder of the swab device and mounted onto a curved die plate (203) of a punch apparatus (200) having a punch (201), a punch guide (202) and a die plate (203). Since the sample cartridge has an aperture in the support, a disc (204) can be punched directly from the sample and be released below the die plate, for example into a well of a sample plate, such as a microtitre plate.

The apertures present in the sample cartridge may be designed to have a size compatible with and/or corresponding to, the size of sample it is desired to punch from the media, such as from 1 to 10 mm, e.g. 2 to 8 mm

In the various aspects of the invention, the die plate can, for example, be convex, concave or substantially planar, depending on the nature of the head/cartridge and, in the case of curved media, whether the sample is to be processed in an inverted or upright configuration.

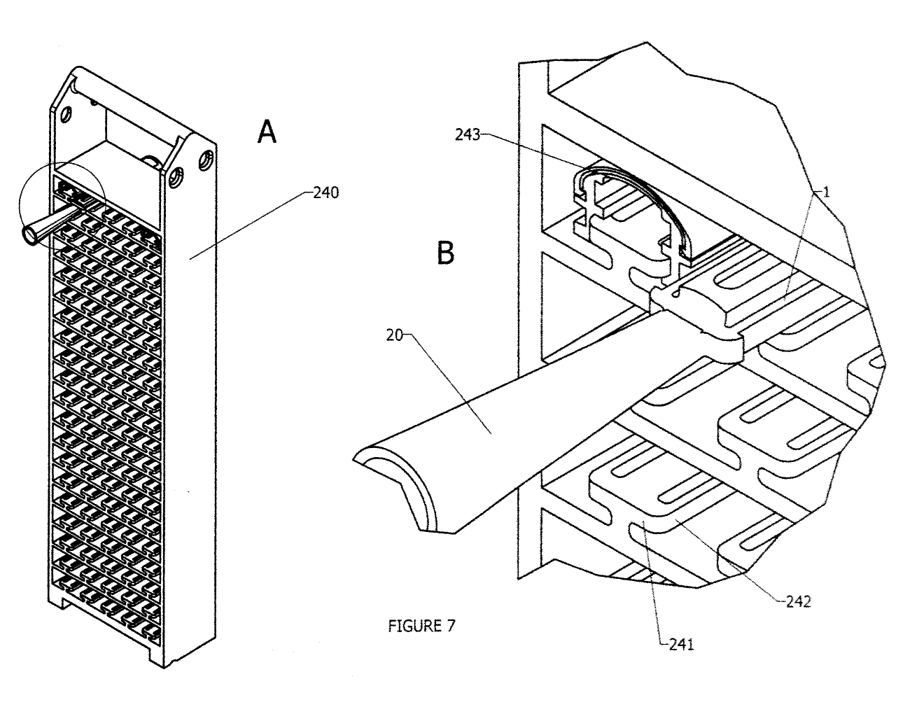

The sample cartridges/cassettes may also be transferred to storage. In one embodiment, an example of which is shown in FIG. 7, the storage comprises a magazine (240) with a plurality of sample loading positions (241). The magazine (240) may have, for example, at least 10 sample loading positions, such as at least 20 or 50 sample loading positions. For example, the number of loading positions may correspond to the number of sample processing positions or wells in subsequent stages of processing e.g. 96 well microtitre plates, a commonly used format. Each loading position (241) comprises a sample engagement means (242) suitable for engaging with a corresponding region of the sample cartridge (243). The neck portion (20), may optionally be subsequently detached leaving only the sample cartridge (243) engaged in the loading position (241). Alternatively, the neck portion (20) may be retained as shown in FIG. 7).

The magazine arrangement and the design of the head/cartridge allow easy transfer and storage of multiple samples.

Accordingly, the present invention also provides a storage unit or magazine for storing sample cartridges/sample cassettes of the invention comprising a plurality of sample loading positions adapted to engage a sample cartridge/sample cassette of the invention. In one embodiment, the storage unit is pre-loaded with a plurality of sample cartridges/sample cassettes of the invention.

The storage unit can also be used more broadly in relation to swab devices in addition to those described above, including the swab devices head described in the third aspect of the invention. The storage unit can serve a number of functions.

Firstly, it serves as a convenient means by which the various component inputs of the system, whether using a direct application form of the invention (direct application system), or using a transfer form of the invention (transfer system) system, can be stored, at the point of collection of samples, both before and after collection of the samples.

Secondly, the unit/magazine serves as the storage and transportation means for the media in higher throughput situations. The magazines will typically need to be securely held when heads/cartridges containing samples are to be loaded into the magazines, from the swab devices. This will also be the case in situations where the magazines are pre-loaded with un-used heads/cartridges before sample collection. In that case, an end portion of the swab device, such as a neck portion, will be inserted into the magazine, and the head/cassette engaged, and withdrawn from the magazine.

Preferred elements of the magazine include the heads/cartridges being held securely within the magazine, preferably when the magazine is placed in any orientation, and that there is minimal opportunity for cross contamination of samples. Preferably the magazine has features that allow it to be used as an input magazine for the further automated processing of the sample. Thus, the magazine is designed such that an automated device could remove the heads/cartridges from the magazine separately, and return them to the same place in the magazine after processing. Examples of suitable designs in this respect have been described above in relation to the sample cartridges of the present invention. Nonetheless, the magazine can be designed to be loaded and unloaded using manual methods.

In a third aspect, the present invention is directed to a head for a swab device which head includes a plurality of discrete, removable portions of swab material which together form a castellated surface.

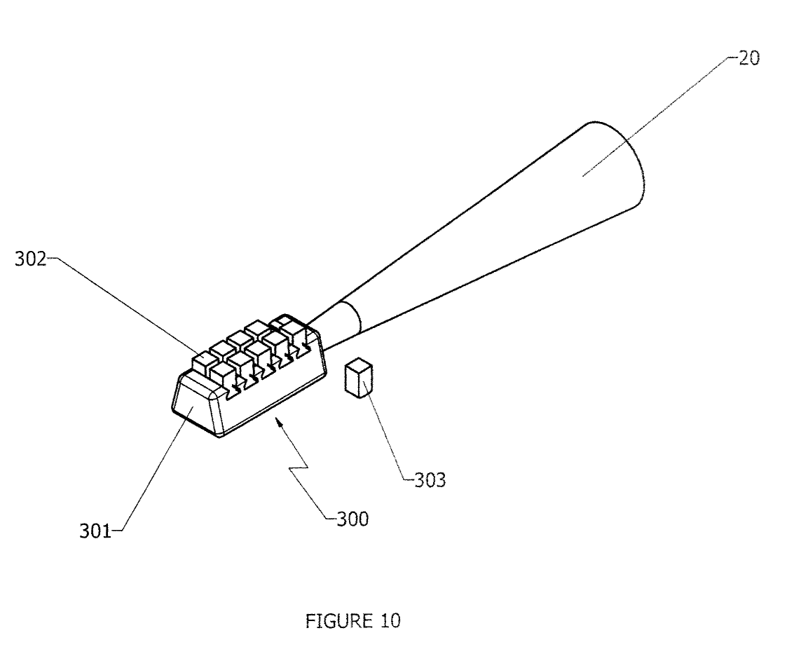

Referring to FIG. 10, the head (300) of the device comprises a support (301) within which number of small pieces of media (302), such as filter paper, are locked in one or more rows to form a castellated surface. In this way, a number of the individual pieces of media each collect biological material, such as a buccal sample, at the same time when the sample is collected.

The individual pieces of media can then be removed independently, using automated or manual methods. For example, a rod can be used to push one or more of the small pieces of media locked into the head, so that the piece that is pushed out can then fall under gravity or by a controlled means, so as to be located into the receiving well of a laboratory tray in which the sample is to be further processed.

The push rod may be disposable, and the part of the push rod that has come into contact with the sample may be automatically or semi-automatically removed and disposed of and replaced by an alternative push rod.

Separate pieces of media, each of the same size, allows for the head to be accessed multiple times for sample.

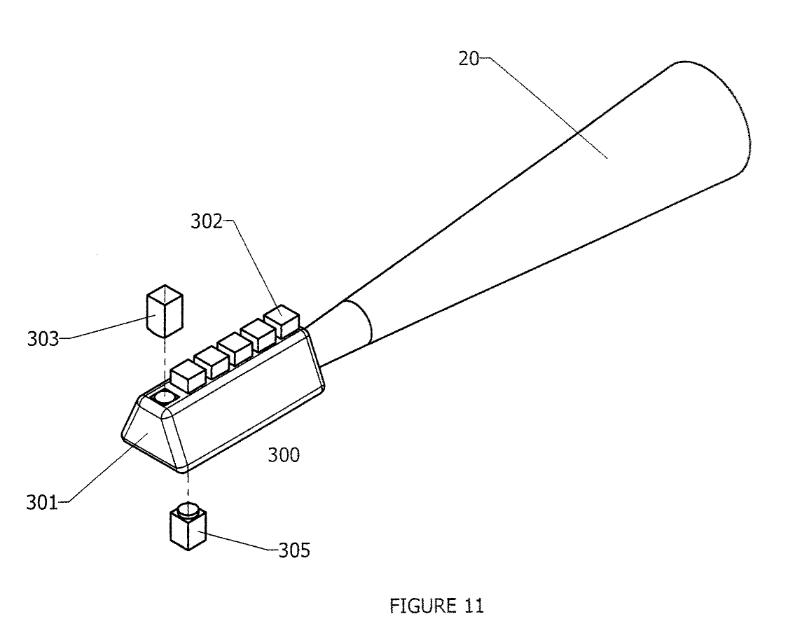

In a particular embodiment shown in FIG. 11, wherein each piece of sample material/media block (302) is mounted in the head (301) over a pin (305) which allows each portion to be independently removed from the head by displacement of the corresponding pin. The ejector pin may remain in the head or displaced. The ejected media block (303) can then be further processed.

The swab material is preferably a cellulose based material, such as filter paper.

In one embodiment, the base of the block of swab material comprises a composition for protecting genetic material in the sample from degradation, as described above.

Typically, the uppermost surface of each portion of swab material may have a surface area of from 1 to 30 mm².

The heads of this third aspect of the invention can also be designed for use with the storage units described above for the second aspect of the invention.

In the fifth aspect, the present invention provides a head for a swab device, which head comprises a solid support comprising one or more apertures, such as a plurality of apertures (e.g. from 2 to 6 apertures), and a layer of swab material overlaying the one or more apertures. The solid support may be substantially planar or curved (e.g. a convex curve as described above in the second aspect of the invention). A slight convex curve may assist particularly in obtaining buccal samples (e.g. φ as defined above in the second aspect is typically from about 5 to 25 degrees, such as from 10 to 20 degrees). The solid support may be rigid or flexible, but is preferably substantially rigid. In one embodiment, however, it is preferred that the solid support has sufficient flexibility to enable for head to adopt the shape of the surface from which the sample is being taken, e.g. the inside of the cheek.

In one embodiment the solid support has a thickness of 5 mm or less, such as from 1 to 5 mm, e.g. from 1 to 2 or 1 to 3 mm.

In one embodiment, the support has a width of from 3 to 10 mm, such as from 3 to 6 mm.

As described above in relation to the second aspect of the invention, the apertures are provided to enable direct punching out of samples of media through the head e.g. by placing the head on a die plate so that at least one of the apertures in the head is aligned with an aperture in the die plate (FIG. 5). The apertures present in the head may be designed to have a size compatible with and/or corresponding to, the size of sample it is desired to punch from the media, such as from 1 to 10 mm, e.g. 2 to 8 mm.

Accordingly the layer of swab material is mounted over the solid support so that the material overlay the apertures. The swab material can be any suitable material, such as is described in the section above entitled “Swab materials”. Preferably the swab material is a spongy material, such as a soft sponge material, a porous (poly)urethane foam, a polyurethane open cell foam or a medical grade foam such as Microbisan™.

A porous swab material may be selected so that it is capable of capturing cellular material, i.e. whole cells. Preferably the porous material has an average pore size of at least 20 μm, more preferably at least 50 or 100 μm. Typically the average pore size is less than 1 mm, such as less than 500 or 200 μm.

Typically, the layer or layers of swab material are relatively thin, having a total thickness of 5 mm or less, e.g. less then 4 or 5 mm, such as from 0.1 to 5 mm or from 1 to 3 mm, typically at least 0.3 or 0.5 mm.

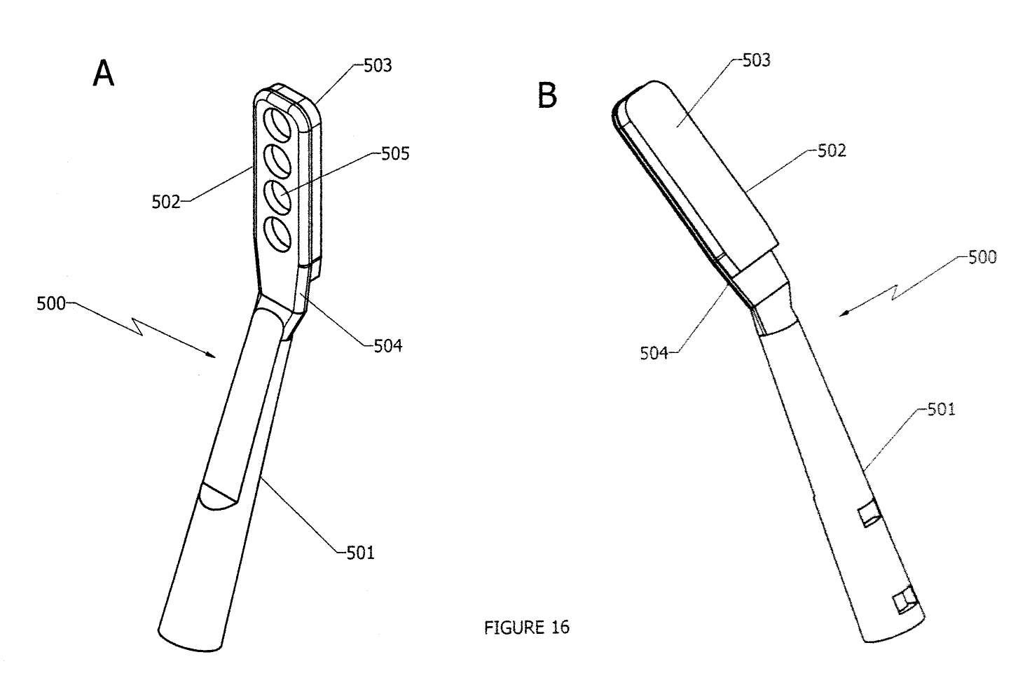

The head may be of any suitable shape, such as substantially rectangular, substantially square and the like. Preferably the head is elongated (FIG. 16). An elongate head has the advantage that where a flexible support is used, the head need only flex/bend in one dimension to adopt the profile of the surface from which the sample is being taken.

In one embodiment, the head is fixed to a neck portion to form a head assembly (FIGS. 14 to 16). In a particular embodiment, the head is at an oblique angle to the neck (see FIGS. 14 to 16), such as an angle of from 135 to 175 degrees, for example 145 to 160 degrees.

Exemplary embodiments are shown in FIGS. 14 to 16. In FIG. 14, the head assembly (500) has a neck portion (501) and a head (502), the head being inclined to the neck. The head includes a swab material (503) mounted over a solid support (504), the head is elongated/rectangular. This type of swab is typically used in the transfer format described above.

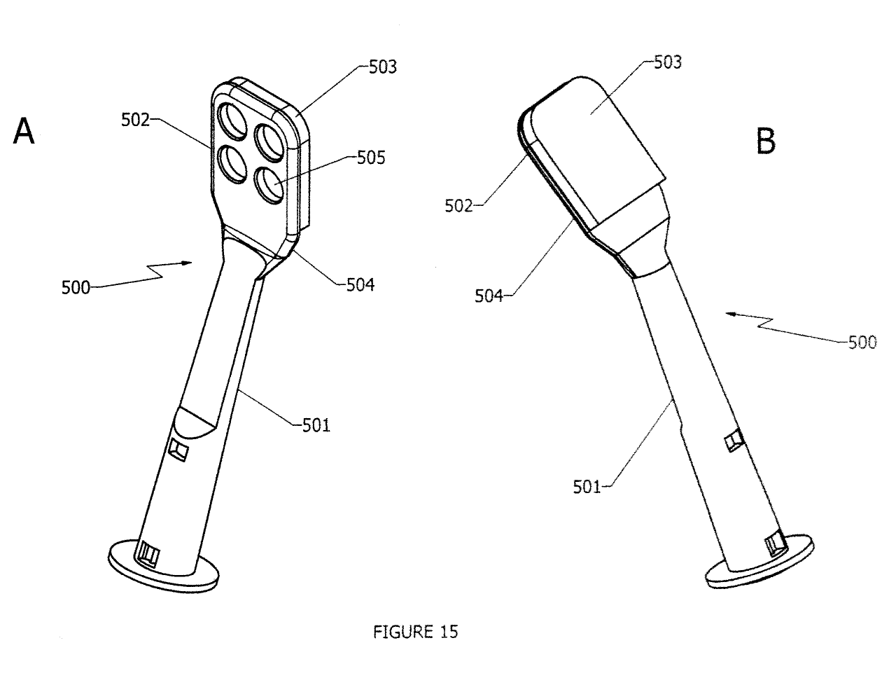

In FIG. 15, the solid support (504) includes four apertures (505) through the solid support, but not the swab material (503). The sample can be processed by placing the head in a die and punching out a portion of the swab material (503) through one or more of the apertures (505). This type of swab is typically used in the direct application format described above.

The swab head assembly shown in FIG. 16 is similar to FIG. 15 except that the head is elongated and the apertures (505) are in a linear arrangement from the tip to the base of the head.

The swab head and swab head assembly are typically used with the swab device of the first aspect of the invention.

In any of the aspects of the present invention it may desirable for an identification tag, such as a barcode, to be printed on or otherwise attached to the media for sample identification purposes.

Supply and Use of Components

The powered or other handle of the swab device of the first aspect may be provided to the user, such as a collection centre, separately from the disposable elements of the device, as can the storage unit of the second aspect where applicable. Storage unit magazines may be supplied pre-loaded with the heads (e.g. cartridges with or without other portions of the swab device), in higher throughput situations.

Kits can be provided to the user can include the neck portion of the device, and guard where applicable, as well as other items such as gloves, ethanol wipes etc.

In smaller throughput cases, where heads/sample cartridges are not provided already loaded into magazines, they can be supplied to the user with the sample collection kit, and in that case, will have been wrapped or covered to prevent contamination from or contact with any other surface. In some cases, complete, individual swab devices can be supplied with disposable handle, integral guard, neck portion (integral or removable) and optionally a sample cartridge, in kit form or pre-assembled and packaged to protect from environmental contamination.

In one embodiment, if the samples are to be processed using semi-automated methods, then after the sample is taken, the head/cartridge and the neck portion are not separated, but are forwarded together for processing, such that the neck portion can form a convenient method whereby the head can be manually held during processing steps such as punching out of pieces of the sample.

The punch and die set of any equipment for semi-automated processing of the sample would be shaped so that the sample is supported adjacent to a die, and the punch moved through the middle section of the head/cartridge, striking the media, and punching out the disk into and through the die. One exemplary embodiment of this is set out in FIG. 5. Other methods of removing small sections of the sample for further processing are also envisaged, such as the use of laser cutting.

If however the samples are to be processed using automated means, then the head/cartridge can be inserted into a magazine, optionally held within a work station at the collection point. The neck portion will generally be removed as part of the process of loading the head/cartridge into the magazine. The magazine and the process of loading the head/cartridge into the magazine are designed to minimize the potential for the head/cartridge to come into contact with any other surface. The magazine is preferably designed to be an integral part of the system, and acts as the input magazine for the automated punch.

The work station may also include provision for storage of preloaded magazines, magazines filled with used heads/cartridges, powered handles and/or collection kits (e.g. guards, sterile wipes, gloves etc).

In a preferred embodiment, the swab device comprises media on a head that is attached to a neck portion, which is in turn attached to a handle, where there is a disposable circular guard between the neck portion and handle.

In some embodiments, particularly in relation to the first aspect of the invention, the collected sample is to be transferred to storage media rather than processed directly. In one embodiment, using the transfer method, the swab media, termed transfer media, that is attached to the head is made of a soft sponge material, which may be suitably shaped to readily collect buccal sample.

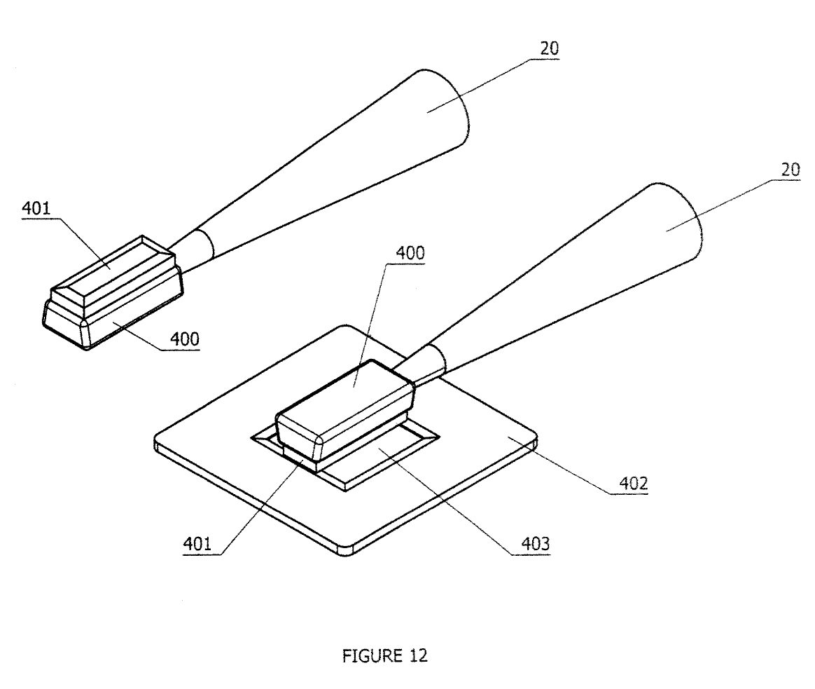

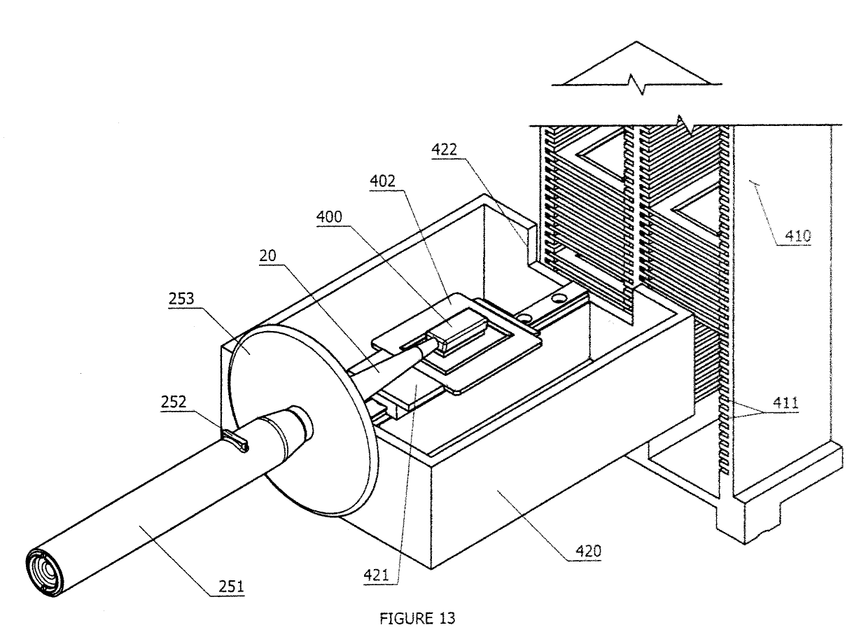

Referring to FIGS. 12 and 13, the swab device comprises a head (400) fixed to a neck portion (2). A sponge material forms the swab material/transfer media (401). Once a sample has been collected onto the swab material, the swab device is taken to a transfer station (420) which includes a support means (421) to hold the storage medium (402, 403) whilst transfer takes place. The storage medium (402, 403) may take the form of a support (402) within which is mounted material (403) for storage of the biological sample. Following contact of the swab material/transfer media (401) with the storage material (403), the powered handle (251) is operated to cause vibration and/or rotation of the head (400). After sufficient time has elapsed for sample transfer, the storage medium (402, 403) can be transferred to storage. FIG. 13 shows a particular embodiment where the transfer station (420) includes an opening (422) which is aligned with one of a plurality of sample loading positions (411) of a magazine/storage unit (410). Once transfer of sample has occurred, the storage medium (402, 403) is slid across via the opening and into a free sample loading position. The transfer station (420) and/or the magazine/storage unit (410) are then moved so that the transfer station (420) is aligned with a new free sample loading position. The new free sample loading position may contain a blank, i.e. unused, storage medium (402, 403) which is moved out of the magazine/storage unit (410) and into position within the transfer station (420).

While it is envisaged that sample could be transferred from the collection media to a long term storage without the use of a transfer station, this process can be enhanced with the use of a transfer station, where that station is designed to include a place for inserting the media that is to form the long term storage for the sample, and a mechanism for locking the head/cartridge and collection media into a position such that there is pressure between the collection media, and the long term storage media. When the collection media is locked into this position, the handle and neck portion are still attached to the head/cartridge. The design is preferably such that when the handle is allowed to vibrate, or otherwise move the head relative to the body of the device, there is movement of the collection media against the long term storage media, thus causing some of the sample to be transferred from the collection media to the long term storage media.

In one embodiment, the long term storage media is secured within a cassette, which provides a secure and stable frame for the media.

In order to maximize the amount of sample transferred from the collection media to the long term storage media, while at the same time not damaging the long term storage material, it is possible to adjust the amount of pressure between the two media, the frequency of the vibration of the handle, and the period of vibration. Typically, the collection media is applied to the long term storage media for a period of from 5 to 20 seconds.

After the transfer has taken place, the user has the option of using a representative part of the sample that remains on the collection media for its assay purposes, using either manual or automated methods of removing and testing that part of the sample, or of using a part of the sample that has been transferred to the long term storage media for that assay, again by separating a representative part of the sample using manual or automated methods.

Once it is satisfied that sample has been effectively transferred from the collection media to the long term storage media, the laboratory has the option of either keeping in storage, or discarding the collection media.

Typically, the long term storage media is re-located from the transfer station to a magazine (as described above), either manually or automatically. The magazine may be the input magazine designed for the punching/sampling apparatus that would later be used to separate a representative piece of the sample for processing.

This could also be the case in the event that the laboratory decides to store the collection media for later use.

An example format for the direct application system and the transfer system in relation to the collection of buccal samples is now described:

In the direct application form, the system can comprise:
1) A head/cartridge or other device of a size suitable for placing in the human mouth comfortably which incorporates long term storage media onto which the sample will be collected;
2) A neck portion to hold the head/cartridge or other device, so that the head/cartridge or other device can be comfortably placed in the human mouth;
3) A guard to protect the hand of any person who is holding the device, as part of the process of collecting buccal sample from the mouth of a sample donor;
4) A powered device to which the neck portion can be connected which causes the neck portion (and therefore the head/cartridge) to vibrate or rotate, and thereby make the process of collecting buccal samples easier; and
5) A magazine into which the head/cartridge can be loaded into and separated from the remainder of the swab device, including the neck portion, if necessary, for the purpose of storage, transport, and later processing;

Components 1 to 4 constitute the swab device.

In the transfer form, the system can comprise:
1. A head/cartridge or other device suitable for placing in the human mouth comfortably to which the selected collection material is attached;
2. A neck portion to hold the head/cartridge or other device, so that the head/cartridge or other device can be comfortably placed in the human mouth;
3. A guard to protect the hand of any person who is holding the device, as part of the process of collecting buccal sample from the mouth of a sample donor;
4. A powered device to which the neck portion can be connected which causes the neck portion (and therefore the head/cartridge) to vibrate or rotate, and thereby make the process of collecting buccal samples easier;
5. A transfer station into which the head/cartridge can be placed and at least some of the sample transferred to long term storage media.
6. Provision for the storage of the long term storage media, in magazines.

Components 1 to 4 constitute the swab device.

The various features and embodiments of the present invention, referred to in individual sections above apply, as appropriate, to other sections, mutatis mutandis. Consequently features specified in one section may be combined with features specified in other sections, as appropriate.

Various modifications and variations of the described methods and products of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the relevant fields are intended to be within the scope of the following claims.

We claim:

1. A swab device for collecting a biological sample from an individual, the swab device comprising a head having a swab material for trapping a biological sample thereon and a mechanism for moving the head relative to the body.

2. The swab device of claim 1 wherein the mechanism is adapted to vibrate and/or rotate the head.

3. The swab device of claim 1 wherein the biological sample comprises genetic material.

4. The swab device of claim 1 wherein the swab material comprises a solid matrix selected from the group consisting of cellulose based material, hydrophilic polymers, synthetic hydrophilic polymers, polyester, polyamide, carbohydrate polymers, polytetrafluoroethylene, fibre glass, porous ceramics and soft spongy materials.

5. The swab device of claim 1 wherein at least the portion of the head comprising the swab material is removable from the body.

6. The swab device of claim 1 wherein the device comprises a guard interposed between the head portion and a portion of the body for avoiding contamination of the portion of the body below the guard.

7. The swab device of claim 6 wherein the guard is removable from the body.

8. The swab device of claim 6 wherein the guard is integrally formed with the head.

9. A method of obtaining a biological sample from an individual which method comprises the steps of (i) providing a swab device comprising a head having a swab material for trapping a biological sample thereon and a body comprising a mechanism for moving the head relative to the body; and (ii) contacting at least a portion of the head with a surface of the individual, the head of the swab device being moved relative to the body of the swab device whilst the head of the device is in contact with the surface.

10. The method of claim 9 wherein the head of the swab device is vibrated and/or rotated by the body of the swab device whilst the head of the device is in contact with the surface.

11. The method of claim 9 wherein the surface of the individual is an interior surface.

12. The method of claim 9 which further comprises the step of (iii) transferring at least a portion of the sample present on the head of the device to a storage medium by contacting the storage medium with the swab material.

13. The method of claim 12 wherein the head of the device is moved relative to the body of the device when the swab material is in contact with the storage medium.

14. The method of claim 12 wherein the head of the device is vibrated and/or rotated by the body of the device when the swab material is in contact with the storage medium.

15. The method of claim 12 wherein the storage medium comprises a composition for protecting genetic material in the sample from degradation.

16. A sample cartridge for a swab device which sample cartridge comprises a sheet of swab material having a convex surface, which material is capable of trapping a biological sample when the convex surface is contacted with a surface of an individual, and a support onto which the swab material is mounted.

17. A sample cartridge for a swab device which sample cartridge comprises a sheet of flexible swab material capable of trapping a biological sample when the material is contacted with a surface of an individual, and a substantially planar flexible support onto which the swab material is mounted, the support comprising means for reversibly engaging the sample cartridge with an end portion of a swab device, the sample cartridge being capable of adopting a convex configuration when engaged with the end portion of the swab device.

18. A sample cassette for the sample cartridge of claim 16 which cassette comprises a substantially planar sheet of a swab material capable of trapping a biological sample when the swab material is contacted with a surface of an individual, wherein two opposing sides of the sheet are each attached to an elongate member, the elongate members being capable of engaging the support of the sample cartridge so as to secure the sheet to the support with the sheet adopting a convex configuration.

19. A sample cassette for a swab device which cassette comprises a substantially planar sheet of a swab material capable of trapping a biological sample when the swab material is contacted with a surface of an individual, wherein two opposing sides of the sheet are each attached to an elongate member, the elongate members being capable of engaging an end portion of a swab device so as to secure the sheet to the end portion with the sheet adopting a convex configuration.

20. A swab device head which comprises a plurality of discrete, removable portions of swab material which portions together form a castellated surface.

21. A swab device head which comprises a layer of porous material capable of trapping cells, overlaying, and in contact with, a layer of cellulose-based material.

22. A head for a swab device according to claim 1 which head comprises a solid support comprising one or more apertures and a layer of swab material overlaying the one or more apertures.
